(19) United States
(12) Patent Application Publication
(10) Pub. No.: US 2001/0044414 A1
(43) Pub. Date: Nov. 22, 2001

(54) Metastasis genes and uses thereof

(75) Inventors: Edwin A. Clark, Ashland, MA (US); Todd R. Golub, Newton, MA (US); Richard O. Hyness, Winchester, MA (US); Eric S. Lander, Cambridge, MA (US)

Correspondence Address: Doreen M. Hogle, HAMILTON, BROOK,, SMITH & REYNOLDS, P.C., Two Militia Drive, Lexington, MA, 02421-4799, US

(73) Assignee: Whitehead Institute for Biomedical Research, Cambridge, MA (US)

(21) Appl. No.: 09/735,273

(22) Filed: Dec. 11, 2000

Related U.S. Application Data
(60) Provisional application No. 60/170,233, filed on Dec. 10, 1999.

(51) Int. Cl. A61K 48/00; C12Q 1/68; G01N 33/574

(52) U.S. Cl. 514/44; 435/6; 435/7.23

(57) ABSTRACT

The identification of a subset of genes which function in metastasis of tumor cells is described. Also described are methods of diagnosis and therapy of metastatic conditions relating to the identified genes.

### RELATED APPLICATIONS

This application claims the benefit of U.S. Provisional Application Serial No. 60/170,233 filed Dec. 10, 1999, the entire teachings of which are incorporated herein by reference.

### GOVERNMENT SUPPORT

The invention was supported, in whole or in part, by grant RO1-CA17007 from the National Cancer Institutes. The Government has certain rights in the invention.

### BACKGROUND OF THE INVENTION

Metastasis, the process whereby tumor cells migrate throughout the body, is complex. In order for a tumor to produce metastases it must contain cells of the correct genotype be capable of completing a complex series of steps. The steps of tumor cell metastasis include the detachment of tumor cells from the primary neoplasm, invasion into the surrounding stroma, intravasation into the vasculature or lymphatic system, survival in the circulation, extravasation into the new host organ or tissue, and then survival and growth in this new microenvironment (Van Noorden et al., 1988). Specific genes are likely to control specific events at each of these steps; however, to date, relatively few genes have been implicated in the process of tumor metastasis. Nm23, KiSS1, CD82/KAI1, E-cadherin, and thrombospondin 1 have been identified as genes capable of suppressing metastasis in various experimental tumor models (Fidler and Radinsky, 1966; Roberts, 1996), while ras, CD44, thymosin β15, and Tiam1 are among the genes capable of inducing metastasis (Vousden et al., 1986; Sherman et al., 1994; Bao et al., 1996; Habets et al., 1994). While these studies have enhanced the understanding of metastasis, they provide only a partial picture of such a complex system.

### SUMMARY OF THE INVENTION

Cancer cells must complete several sequential steps to produce the metastases that cause the majority of deaths from this disease. Using an in vivo selection scheme to select for highly metastatic tumor cells, a global gene expression analysis of metastatic tumors generated by human and mouse melanoma cells was performed. Of the over 6000 human and mouse genes examined, only 32 genes were consistently and significantly altered; one-third of these genes regulate, either directly or indirectly, the actin-based cytoskeleton. One of these genes, the small GTPase rhoC, enhances metastasis when overexpressed, while a dominant-negative rho inhibits metastasis. Analysis of the phenotype of the dominant-negative rho- and rhoc-expressing cells suggests an important role for rhoC in tumor cell invasion. This finding confirms the results of the genomic screen and indicates a role for cytoskeletal organization in tumor metastasis.

The present invention relates to genes which function in the regulation of tumor cell metastasis, particularly those genes which regulate the actin-based cytoskeleton of tumor cells. Work described herein provides methods of screening for agents which affect metastasis, particularly with respect to the metastasis genes identified as described herein, as well as diagnostic and therapeutic methods relating to these genes and their encoded gene products.

Thus, the invention relates to a method of inhibiting metastasis in a mammal, e.g., a human, comprising administering to a mammal in need thereof an effective amount of an agent which alters the actin-based cytoskeleton of one or more cells in the mammal. In one embodiment, the agent inhibits formation of the actin-based cytoskeleton. In a particular embodiment, the agent inhibits the activity of a gene selected from the group consisting of the genes encoding fibronectin, RhoC, thymosin β4, t-PA, angiopoietin 1, IEX-1/Glu96, RTP/NDR1, fibromodulin, Hsp70, IL13 Rec. α2, Sec61β, snRNP polypeptide C, collagen Iα2, UBE21, KIAA0156, TGFβ superfamily, surfactant protein C, lysozyme M, matrix Gla protein, Tsa-1, collagen IIIα1, biglycan, α-catenin, valosin-cont. prot., ERK-1, α-actinin 1, calmodulin, EIF4γ, α-centractin, IQGAP1, cathepsin S, EF2, and the genes in Table 5. In another particular embodiment, the agent inhibits the gene encoding RhoC. The agent can inhibit the activity of the gene directly or by inhibiting the activity of a downstream effector of the gene. For example, the agent can be a nucleic acid molecule (e.g., one or more antisense molecules or nucleic acid molecules encoding one or more dominant negative form of a gene product), an antibody, a peptide, an organic molecule, an inorganic molecule, or any combination of two or more of the preceding (e.g., two or more nucleic acid molecules; a nucleic acid molecule(s) and an organic molecules(s)).

The mammal in need of the described treatment can be at risk for a metastatic condition, either genetically (e.g., through heredity) or environmentally, or the mammal can have one or more non-metastatic tumors. For example, the mammal can be at risk for or currently have one or more non-metastatic conditions selected from the group consisting of melanoma, breast cancer, ovarian cancer, prostate cancer, lung cancer, bone cancer, throat cancer, brain cancer, testicular cancer, liver cancer, stomach cancer, pancreatic cancer, and combinations thereof. Thus, the described treatment can be administered prophylactically or therapeutically. The described treatment can also be administered to a mammal having a metastatic condition to inhibit further metastasis.

The invention further relates to a method of predicting the likelihood of development of a metastatic condition in a mammal, e.g., a human, comprising the steps of obtaining a biological sample from a mammal to be tested; determining the level of one or more gene products which alter the actin-based cytoskeleton of one or more cells in the mammal (i.e., the test level); and comparing the test level with an appropriate control, wherein if the test level is greater than the level of the gene product in a normal sample, then the mammal has an increased likelihood of developing a metastatic condition. The control can be a sample from a normal mammal or a sample from a mammal having a metastatic condition.

In one embodiment, the gene product is selected from the group consisting of fibronectin, RhoC, thymosin β4, t-PA, angiopoietin 1, IEX-1/Glu96, RTP/NDR1, fibromodulin, Hsp70, IL13 Rec. α2, Sec61β, snRNP polypeptide C, collagen Iα2, UBE21, KIAA0156, TGFβ superfamily, surfactant protein C, lysozyme M, matrix Gla protein, Tsa-1, collagen IIIα1, biglycan, α-catenin, valosin-cont. prot., ERK-1, α-actinin 1, calmodulin, EIF4γ, α-centractin, IQGAP1, cathepsin S, EF2, and the genes in Table 5. In a preferred embodiment the gene product is RhoC.

In one embodiment, the metastatic condition is selected from the group consisting of metastatic forms of melanoma, breast cancer, ovarian cancer, prostate cancer, lung cancer, bone cancer, throat cancer, brain cancer, testicular cancer, liver cancer, stomach cancer, pancreatic cancer, and combinations thereof.. In another embodiment, the biological sample is a blood sample or a cell sample from a tumor in the mammal.

The invention further relates to a method of identifying an agent which regulates metastasis of a tumor cell, comprising the steps of contacting one or more tumor cells with an agent to be tested; and determining the level of one or more gene products which alter the actin-based cytoskeleton in the cell, wherein if the level of the gene product is altered in the presence of the agent as compared with the level of the gene product in the absence of the agent, then the agent regulates metastasis of the tumor cell. In one embodiment, the gene product is selected from the group consisting of fibronectin, RhoC, thymosin β4, t-PA, angiopoietin 1, IEX-1/Glu96, RTP/NDR1, fibromodulin, Hsp70, IL13 Rec. α2, Sec6β, snRNP polypeptide C, collagen Iα2, UBE21, KIAA0156, TGFβ superfamily, surfactant protein C, lysozyme M, matrix Gla protein, Tsa-1, collagen IIIα1, biglycan, α-catenin, valosin-cont. prot., ERK-1, α-actinin 1, calmodulin, EIF4γ, α-centractin, IQGAP1, cathepsin S, EF2, and the gene products listed in Table 5. The invention also relates to a method of inhibiting metastasis in a mammal, comprising administering to a mammal in need thereof an effective amount of an agent which alters the actin-based cytoskeleton of one or more cells in the mammal, wherein the agent is identified by this method.

In an alternate embodiment, the present invention is directed toward a method of inhibiting metastasis in a mammal, comprising administering to a mammal in need thereof an effective amount of an agent which alters the expression of a gene which regulates metastasis in one or more tumor cells in the mammal thereby inhibiting metastasis. In another embodiment, the present invention further relates to a method of predicting the likelihood of development of a metastatic condition in a mammal, comprising the steps of: obtaining a biological sample from a mammal to be tested; determining the level of one or more gene product which regulates metastasis in one or more tumor cells in the mammal; and comparing the level determined in (b) with an appropriate control, wherein if the level determined in (b) is greater than the level of the gene product in said control sample, then the mammal has an increased likelihood of developing a metastatic condition.

The present invention also relates to a method of identifying an agent which regulates metastasis of a tumor cell, comprising the steps of: contacting one or more tumor cells with an agent to be tested; and determining the level of one or more gene products which regulates metastasis in a tumor cell, wherein if the level of the gene product is altered in the presence of the agent as compared with the level of the gene product in the absence of the agent, then the agent regulates metastasis of a tumor cell. The gene product involved in metastasis is selected from the group consisting of fibronectin, RhoC, thymosin β4, t-PA, angiopoietin 1, IEX-1/Glu96, RTP/NDR1, fibromodulin, Hsp70, IL13 Rec. α2, Sec61 β, snRNP polypeptide C, collagen Iα2, UBE21, KIAA0156, TGFβ superfamily, surfactant protein C, lysozyme M, matrix Gla protein, Tsa-1, collagen IIIα1, biglycan, α-catenin, valosin-cont. prot., ERK-1, α-actinin 1, calmodulin, EIF4γ, α-centractin, IQGAP1, cathepsin S and EF2, and the genes in Table 5.

In one embodiment, the present invention relates to a method of identifying an agent which regulates metastasis of a tumor cell, comprising the steps of: contacting one or more tumor cells with an agent to be tested; determining the level of rhoC gene product, wherein if the level of rhoC gene product is altered in the presence of the agent as compared with the level of rhoC gene product in the absence of the agent, then the agent regulates metastasis of a tumor cell.

In another embodiment, the present invention relates to a method of identifying an agent which inhibits metastasis of a tumor cell, comprising the steps of: contacting one or more tumor cells with an agent to be tested; and determining the level of rhoC gene product, wherein if the level of rhoC gene product is decreased in the presence of the agent as compared with the level of rhoC gene product in the absence of the agent, then the agent inhibits metastasis of a tumor cell. In an alternate embodiment, the present invention relates to a method of identifying an agent which increase metastasis of a tumor cell, comprising the steps of: contacting one or more tumor cells with an agent to be tested; and determining the level of rhoC gene product, wherein if the level of rhoC gene product is increased in the presence of the agent as compared with the level of rhoC gene product in the absence of the agent, then the agent increases metastasis of a tumor cell.

The present invention further relates to a method for formulating a therapeutic regimen comprising the steps of: predicting the likelihood of metastasis by a method described herein and formulating the therapeutic regimen accordingly.

### BRIEF DESCRIPTION OF THE DRAWINGS

FIG. 1 illustrates an in vivo selection scheme. Heterogeneous, poorly metastatic melanoma cell lines (human A375P or mouse B16F0) were injected intravenously into the tail veins of host mice, and pulmonary metastases were isolated. These metastases were either minced and grown in tissue culture (to be injected into additional host mice), or RNA was extracted from them to prepare the labeled cRNA target used to hybridize to the oligonucleotide array. The procedure to select for highly metastatic tumor cells was repeated two (for the A375 cells) or three times (for the B16 cells).

FIG. 2A and 2B show that RhoC regulates melanoma cell chemotaxis and invasion. In FIG. 2A, poorly (P) or highly metastatic (M) A375 cells expressing rhoC, rhoA, or dominant-negative (dn) rho were assayed for chemotaxis for 16 hours. Each bar represents the mean ± SEM of four experiments done in duplicate. In FIG. 2B, the cell lines described above were assayed for invasion for 48 hours. Each bar represents the mean ±SEM of three experiments done in duplicate.

### DETAILED DESCRIPTION OF THE INVENTION

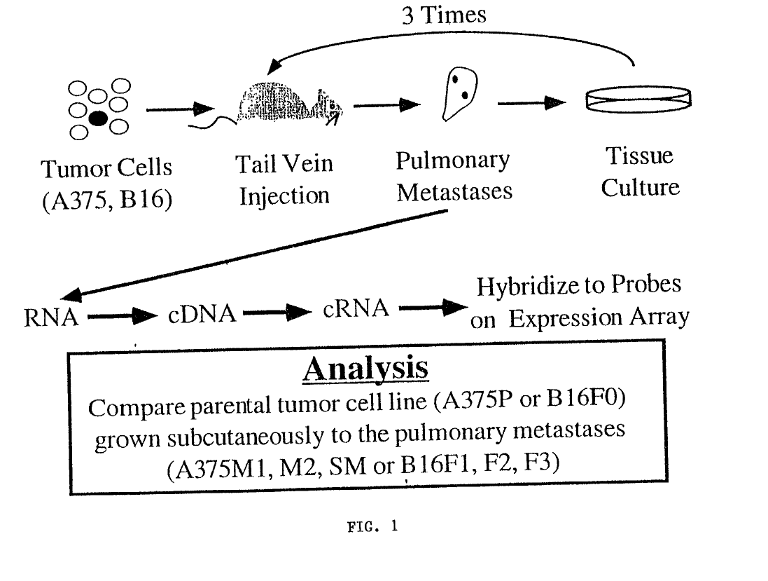

Metastasis is the principal cause of death from cancer. Recent advances in genomic research allow the functional mapping of genes involved in complex processes such as metastasis. The present invention encompasses a comprehensive molecular characterization of metastasis by determining the expression patterns of several thousand genes simultaneously using oligonucleotide microarrays (Fodor, 1997). This genomic approach allowed rapid analysis of the gene expression profile in metastatic tumors, providing insight into the genetic blueprint that allows tumors to metastasize. While the power of the genomics approach is that it can analyze and identify thousands of genes whose expression is altered between two samples, comparison of two radically different samples does not provide the best information, since there are likely to be many differences in gene expression that are not related to the phenotypic or functional difference of interest. Therefore, it was essential to define an experimental system in which the only difference between two samples was the ability to metastasize. Melanoma is one of the most metastatic cancers (and therefore one of the most deadly) in humans. As shown in FIG. 1, the model system used as described herein involves the in vivo selection of highly metastatic melanoma cells out of a heterogeneous population of poorly metastatic tumor cells (Fidler, 1973). Two different melanoma cell lines, the human A375 and the mouse B16, were examined to identify a subset of genes that are important for all melanomas to metastasize, not just melanomas of a specific cell lineage.

An additional goal of these studies was the identification of metastasis-specific genes. There are several reasons to believe that specific gene products are capable of regulating metastasis without altering the growth properties of a tumor. First, both metastatic and poorly metastatic melanoma cells are capable of producing subcutaneous tumors. Second, transfer of specific chromosomes to metastatic melanomas suppresses their ability to metastasize without affecting tumorigenicity (Welch et al., 1994). The results described herein support the hypothesis that metastasis is due to the activation (or inactivation) of genes that regulate one or more steps of metastasis. The work described herein provides evidence that enhanced expression of several genes that regulate cytoskeletal organization parallels the emergence of a metastatic phenotype, and that one of these genes, the small GTPase rhoC, is necessary and sufficient for metastasis. Finally, work described herein supports a role for rhoC in promoting metastasis by enhancing cellular properties required for intravasation/extravasation. As used herein, intravasation is defined as the movement or migration of a cell into the vasculature or lymphatic system. As used herein, extravasation is defined as the movement or migration of a cell out of the vasculature or lymphatic system and into an organ or tissue.

During cancer progression, the most damaging alteration that takes place in a tumor cell is the switch from a locally growing tumor cell to a metastatic tumor cell. As described herein, global gene expression analysis showed a pattern of gene expression that correlates with the progression to a metastatic phenotype; in particular, enhanced expression of several genes that regulate, either directly or indirectly, the actin-based cytoskeleton was identified. The actin-based cytoskeleton, as used herein, refers to microfilaments and their associated proteins which are a part of the cell architecture.

RhoC, one of the cytoskeletal regulators identified in this genomic screen is, as described herein, essential for tumor metastasis. The cytoskeleton is composed of fibers comprising microfilaments, intermediate filaments, and microtubules which are important in cell structure, differentiation, and movement. The observation that expression of a single gene is sufficient to induce metastasis is surprising, given that metastasis is such a complex process.

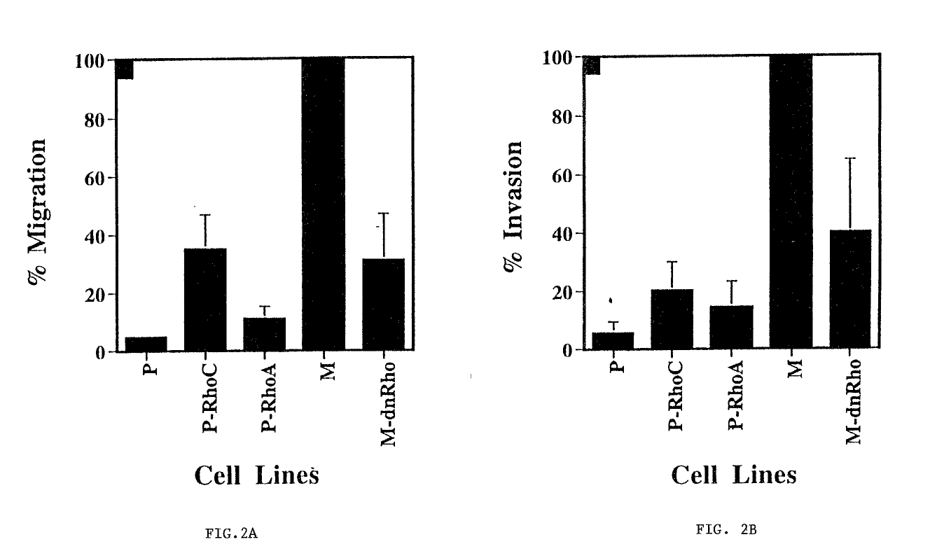

RhoC is a member of the Rho GTPase family that has been shown to regulate the actin cytoskeletal organization in response to extracellular factors (van Aelst and Dsouze-Schorey, 1997). When compared to rhoA, the canonical family member, relatively little is known about rhoC. RhoA and RhoC are highly homologous, with only six non-conservative amino acid substitutions, all in the C-terminal end of the molecules. Since the sequence of the N-terminus of Rho proteins, which is likely to harbor their putative effector domain, is conserved, it is likely that the molecules that act downstream of rhoA and rhoC are also conserved. Among the potential effector molecules for rhoC in regulating the actin cytoskeleton is the rho-associated kinase ROCK. ROCK was recently shown to enhance tumor cell invasion in an in vivo assay (Itoh et al., 1999), implicating it in events that may be essential for metastasis. Therefore, it is logical to hypothesize that rhoA should be capable of enhancing tumor metastasis. However, as described herein, rhoA is expressed at equivalent levels in both the poorly- and highly-metastatic tumors, suggesting that rhoA expression is not sufficient for metastasis. Furthermore, when rhoC and rhoA were expressed at equivalent levels, rhoC was a better mitogen than was rhoA (FIG. 2A-B).

As described herein, vertebrate genes whose expression levels are reproducibly altered in highly metastatic cells have been identified; these genes are referred to herein as “metastasis genes” or genes (or gene products) which “regulate metastasis”. As used herein, a gene which regulates metastasis has been determined by the criteria described herein to be altered in a metastatic (or highly metastatic) cell as compared to its expression in a non-metastatic cell (or poorly metastatic). The expression of the metastasis genes is typically increased in metastatic cells as compared with non-metastatic cells. Many of the metastasis genes which have been identified function in the regulation of the actin-based cytoskeleton. In particular, RhoC has been shown as described herein to be both necessary and sufficient for metastasis. The present invention provides methods of screening for agents which regulate metastasis, particularly with respect to the newly identified metastasis genes, as well as diagnostic and therapeutic methods relating to these genes and their encoded gene products.

Thus, the present invention provides a method of inhibiting metastasis in a mammal, e.g., a human, comprising administering to a mammal in need thereof an effective amount of an agent which alters (e.g., inhibits, enhances or otherwise changes) the actin-based cytoskeleton of one or more tumor cells in the mammal, thereby inhibiting metastasis. As used herein, “alters” refers to a change which can be positive or negative. For example, the cytoskeleton can be altered in such a way that the cell morphology is changed, as described in the Exemplification. In one embodiment, the agent inhibits the actin-based cytoskeleton in tumor cells. In another embodiment, the agent inhibits formation in tumor cells of the elongated morphology described herein to be associated with metastasis. As used herein, inhibition includes any decrease or reduction, both quantitative and qualitative, in the response (e.g., metastasis) or property (e.g., regulation of the actin-based cytoskeleton or elongated cell morphology) to be inhibited, including partial or complete abolishment of the response or property. Additionally, inhibition of metastasis can be a decrease or increase in gene expression of genes involved in metastasis which results in a decrease or prevention of metastasis. For example, inhibition of metastasis by the regulation of one or more metastatic genes described herein. As used herein, “metastasis” is intended to mean the process whereby tumor cells migrate throughout the body (producing metastases). Metastases refers to tumors in a location different from the location of the original tumor.

Mammals which can be treated or diagnosed according to methods described herein include, but are not limited to, primates (e.g., humans), cows, sheep, goats, horses, dogs, cats, rabbits, guinea pigs, rats, mice or other bovine, ovine, equine, canine, feline, rodent or murine species. A mammal to be treated can be at risk for a metastatic condition, either genetically (e.g., through heredity) or environmentally, or the mammal can have one or more non-metastatic tumors. For example, be administered to a mammal having a metastatic condition to inhibit further metastasis, the mammal may be at risk for or currently have one or more non-metastatic conditions selected from the group consisting of melanoma, breast cancer, ovarian cancer, prostate cancer, lung cancer, bone cancer, throat cancer, brain cancer, testicular cancer, liver cancer, stomach cancer, pancreatic cancer, and combinations thereof. Thus, the described treatment can be administered prophylactically or therapeutically. The described treatment can also

In another embodiment, the present invention provides a method of inhibiting metastasis in a mammal comprising the inhibiting the activity of one or more genes selected from the group consisting of the genes encoding fibronectin, RhoC, thymosin β4, t-PA, angiopoietin 1, IEX-1/Glu96, RTP/NDR1, fibromodulin, Hsp70, IL13 Rec. α2, Sec61β, snRNP polypeptide C, collagen Iα2, UBE21, KIAA0156, TGFβ superfamily, surfactant protein C, lysozyme M, matrix Gla protein, Tsa-1, collagen IIIα1, biglycan, a-catenin, valosin-cont. prot., ERK-1, α-actinin 1, calmodulin, EIF4γ, α-centractin, IQGAP1, cathepsin S, EF2, and the genes listed in Table 5. The agent may inhibit transcription of the gene, alter (render non-translatable) or degrade the transcript, or inhibit the activity of the encoded gene product.

Suitable agents can inhibit (i.e., antagonize) the activity of the gene or gene product directly or by inhibiting the activity of a downstream effector of the gene. In a particular embodiment, the gene encodes RhoC; in this embodiment, the agent inhibits rhoC transcription or RhoC activity, or the transcription or activity of downstream effectors of RhoC (see, for example, Ridley et al., Current Biol. 6:1256-1264 (1996)). For example, the agent can be selected from the group consisting of nucleic acid molecules (e.g., one or more antisense molecules or nucleic acid molecules encoding one or more dominant negative form of a gene product), anti-peptide or anti-protein antibodies, peptides (e.g., ligands), organic molecules, inorganic molecules, and combinations thereof. As used herein, a dominant negative form of a gene product refers to a gene product which partially or completely inhibits the function of the target gene. For example, as described in the exemplification, the dominant negative form of rhoA inhibits the activity of its target gene.

Antisense nucleic acids of the invention can be designed using the nucleotide sequences of the gene to be inhibited and constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids.

The term “antibody” as used herein refers to immunoglobulin molecules and immunologically active portions of immunoglobulin molecules, i.e., molecules that contain an antigen binding site that specifically binds an antigen. A molecule that specifically binds to a gene product to be inhibited is a molecule that binds to that polypeptide or a fragment thereof, but does not substantially bind other molecules in a sample, e.g., a biological sample. Examples of immunologically active portions of immunoglobulin molecules include F(ab) and F(ab′)₂ fragments which can be generated by treating the antibody with an enzyme such as pepsin. Both polyclonal and monoclonal antibodies can be suitable agents for use in the methods of the invention, and both can be prepared using methods well known in the art. Additionally, recombinant antibodies, such as chimeric and humanized monoclonal antibodies, comprising both human and non-human portions, which can be made using standard recombinant DNA techniques, are useful in the methods of the invention. Completely human antibodies are particularly desirable for therapeutic treatment of human patients. For a detailed discussion of this technology for producing human antibodies and human monoclonal antibodies and protocols for producing such antibodies, see, e.g., U.S. Pat. No. 5,625,126; U.S. Pat. No. 5,633,425; U.S. Pat. No. 5,569,825; U.S. Pat. No. 5,661,016; and U.S. Pat. No. 5,545,806.

The agent can be formulated in a pharmaceutical composition. For instance, suitable agents can be formulated with a physiologically acceptable medium to prepare a pharmaceutical composition. The particular physiological medium may include, but is not limited to, water, buffered saline, polyols (e.g., glycerol, propylene glycol, liquid polyethylene glycol) and dextrose solutions. The effective amount and optimum concentration of the active ingredient(s) (e.g., the agent) in the chosen medium can be determined empirically, according to procedures well known to medicinal chemists, and will depend on the ultimate pharmaceutical formulation desired. Methods of administration of compositions for use in the invention include, but are not limited to, intradermal, intramuscular, intraperitoneal, intravenous, subcutaneous, intraocular, oral and intranasal. Other suitable methods of introduction can also include rechargeable or biodegradable devices and slow release polymeric devices. The pharmaceutical compositions of this invention can also be administered as part of a combinatorial therapy with other agents.

The invention further relates to a method of predicting the likelihood of development of a metastatic condition in a mammal, e.g., a human, comprising the steps of obtaining a biological sample from a mammal to be tested; determining the level of one or more gene products which alter the actin-based cytoskeleton of one or more tumor cells in the mammal (i.e., the test level); and comparing the test level with an appropriate control, wherein if the test level is greater than the level of the gene product in said control, then the mammal has an increased likelihood of developing a metastatic condition.

For example, the level (i.e., presence, absence or amount) of one or more gene products can be determined by contacting the sample with an antibody which specifically binds to the gene product to be assessed and determining the amount of bound antibody, e.g., by detecting or measuring the formation of the complex between the antibody and the gene product. The antibodies can be detectably labeled (e.g., radioactive, fluorescently, biotinylated or HRP-conjugated) to facilitate detection of the complex. Appropriate assay systems include, but are not limited to, Enzyme-Linked Immunosorbent Assay (ELISA), competition ELISA assays, Radiolmuno-Assays (RIA), immunofluorescence, western, and immunohistochemical assays which involve assaying a particular gene product in a sample using antibodies having specificity for the gene product. Antibodies can also be prepared which bind only to altered forms of the protein, including addition of one or more amino acids, deletion of one or more amino acids or change in one or more amino acids (including substitution of an amino acid for one which is normally present in the sequence). Antibodies can be monoclonal, polyclonal or a mixture thereof. This allows the identification of altered gene products which may alter normal function in cytoskeletal formation and metastasis.

Alternatively, the level of the nucleotide sequence (e.g., DNA or RNA) of the gene in a nucleic acid sample from the mammal can be assessed by combining oligonucleotide probes derived from the nucleotide sequence of the gene with a nucleic acid sample from the mammal, under conditions suitable for hybridization. Hybridization conditions can be selected such that the probes will hybridize only with the specified gene sequence. Alternatively, conditions can be selected such that the probes will hybridize only with altered nucleotide sequences of the gene and not with unaltered nucleotide sequences; that is, the probes can be designed to recognize only particular alterations in the nucleic acid sequence of the gene, including addition of one or more nucleotides, deletion of one or more nucleotides or change in one or more nucleotides (including substitution of a nucleotide for one which is normally present in the sequence). This allows the identification of altered genes which may alter the normal function of the gene product in cytoskeletal formation and metastasis. In a particular embodiment, probes for the metastatic genes described herein can be displayed on an oligonucleotide array or used on a DNA chip, as described in WO 95/11995; such oligonucleotide arrays are within the scope of the invention.

The control can be the level of gene product in a sample from a normal mammal or the level of gene product in a sample from a mammal having the metastatic condition. If the sample is from a normal mammal, then increased levels of the gene product in the test sample compared with the control indicates that the mammal has an increased risk of developing a metastatic condition as compared with the control. If the sample is from a mammal having the metastatic condition, then similar levels of the gene product in the test sample and the control indicates that the mammal has an increased risk of developing a metastatic condition as compared with the control. Alternatively, the level of the gene product in the test sample can be compared with a standard (e.g., presence or absence of gene product) or numerical value determined (e.g.,from analysis of other samples) to correlate with decreased, normal or increased risk of developing a metastatic condition. The advantage of the present invention would be to utilize a more aggressive treatment for a patient at higher risk of a metastatic condition. Correlation can be performed by standard statistical methods such as a Chi-squared test and statistically significant correlations between the regulation of metastasis genes and metastases for a set of individuals which exhibit metastases and a set of individuals which do not.

In one embodiment, the gene product is selected from the group consisting of fibronectin, RhoC, thymosin β4, t-PA, angiopoietin 1, IEX-1/Glu96, RTP/NDR1, fibromodulin, Hsp70, IL13 Rec. α2, Sec61β, snRNP polypeptide C, collagen Iα2, UBE21, KIAA0156, TGFβ superfamily, surfactant protein C, lysozyme M, matrix Gla protein, Tsa-l, collagen IIIα1, biglycan, α-catenin, valosin-cont. prot., ERK-1, α-actinin 1, calmodulin, EIF4γ, α-centractin, IQGAP1, cathepsin S, EF2, and the gene products listed in Table 5. In a particular embodiment, the gene product is RhoC.

The term metastatic conditions, as used herein, includes any conditions or disorders, including, but not limited to, cancer, which are associated with tumor formation. In one embodiment, the metastatic condition is selected from the group consisting of metastatic forms of melanoma, breast cancer, ovarian cancer, prostate cancer, lung cancer, bone cancer, throat cancer, brain cancer, testicular cancer, liver cancer, stomach cancer, pancreatic cancer, and combinations thereof. In another embodiment, the biological sample is a blood sample or a cell sample, e.g., a tumor cell sample, from the mammal.

The invention further relates to a method of identifying an agent which regulates metastasis of a tumor cell, comprising the steps of contacting one or more cells(e.g., a host cell or a tumor cell) with an agent to be tested; and determining the level of one or more gene products which alter the actin-based cytoskeleton in the cell, wherein if the level of the gene product is altered in the presence of the agent as compared with the level of the gene product in the absence of the agent, then the agent regulates metastasis of the tumor cell. The invention also relates to a method of inhibiting metastasis in a mammal, comprising administering to a mammal in need thereof an effective amount of an agent which alters the actin-based cytoskeleton of one or more cells in the mammal, wherein the agent is identified by this method. The step of contacting can be carried out by directly applying the agent to the cell or by combining the agent with a substance which is in contact with the cell (e.g., by administering the agent into cell culture medium). Methods described above for determining the level of gene expression or the level of gene product are also useful in the screening methods of the invention.

In an alternate embodiment, the present invention relates to a method of identifying an agent which regulates metastasis of a tumor cell comprising the steps of contacting one or more cells (e.g., a host cell or a tumor cell) with an agent to be tested; and determining the level of one or more gene products which regulate metastasis in the cell, wherein if the level of the gene product is altered in the presence of the agent as compared with the level of the gene product in the absence of the agent, then the agent regulates metastasis of the tumor cell. As used herein, gene product refers to the DNA, RNA, protein, or fragments, complements, and portions thereof such that the gene product is specific for the gene. Metastasis genes described herein are suitable for use in the present invention. For example, if the level of rhoC gene product is altered in one or more tumor cells, as described in the present invention, in the presence and absence of the agent then the agent regulates metastasis.

The present invention is further directed toward a method of inhibiting metastasis in a mammal, comprising administering to a mammal in need thereof an effective amount of an agent which alters the expression of a gene which regulates metastasis in one or more tumor cells in the mammal thereby inhibiting metastasis. The mammal may be at risk for or currently have one or more non-metastatic conditions. Thus, the described treatment can be administered prophylactically or therapeutically.

In another embodiment, the present invention further relates to a method of predicting the likelihood of development of a metastatic condition in a mammal, comprising the steps of: obtaining a biological sample from a mammal to be tested; determining the level of one or more gene product which regulates metastasis in one or more tumor cells in the mammal; and comparing the level of the metastasis gene in the tumor cell with the level of the metastasis gene in an appropriate control. I f the level of the metastasis gene in the tumor cell is greater than the level of the metastasis gene in an appropriate control, then the mammal has an increased likelihood of developing a metastatic condition.

As described herein, rhoC is necessary and sufficient for metastasis. Therefore, rhoC can be used to identify agents which regulate metastasis. In one embodiment, the present invention is directed toward a method of identifying an agent which inhibits metastasis of a tumor cell, comprising the steps of: contacting one or more tumor cells with an agent to be tested; and determining the level of rhoC gene product, wherein if the level of rhoC gene product is decreased in the presence of the agent as compared with the level of rhoC gene product in the absence of the agent, then the agent inhibits metastasis of a tumor cell. In an alternate embodiment, the present invention relates to a method of identifying an agent which increase metastasis of a tumor cell, comprising the steps of: contacting one or more tumor cells with an agent to be tested; and determining the level of rhoC gene product, wherein if the level of rhoC gene product is increased in the presence of the agent as compared with the level of rhoC gene product in the absence of the agent, then the agent increases metastasis of a tumor cell.

Cells for use in the present invention include cells which naturally express the metastasis genes (e.g., tumor cells) and cells which have been engineered to express the metastasis genes. For example, prokaryotic and eukaryotic host cells can be transfected with expression vectors to express the metastasis genes. Methods for making said cells is routine in the art. Cells which can be transfected with the vectors of the present invention include, but are not limited to , bacterial cells such as E. coli, insect cells (baculovirus), yeast or mammalian cells such as Chinese hamster ovary cells (CHO). Ligating polynucleotide sequences into gene constructs, such as expression vectors, and transforming or transfecting into hosts, either eukaryotic (yeast, avian, insect or mammalian) or prokaryotic (bacterial cells), are standard procedures used in producing proteins.

In one embodiment, the gene product is selected from the group consisting of fibronectin, RhoC, thymosin β4, t-PA, angiopoietin 1, IEX-1/Glu96, RTP/NDR1, fibromodulin, Hsp70, IL13 Rec. α2, Sec61β, snRNP polypeptide C, collagen Iα2, UBE21, KIAA0156, TGFβ superfamily, surfactant protein C, lysozyme M, matrix Gla protein, Tsa-1, collagen IIIα1, biglycan, α-catenin, valosin-cont. prot., ERK-1, α-actinin 1, calmodulin, EIF4γ, α-centractin, IQGAP1, cathepsin S, EF2, and the gene products listed in Table 5.

In one embodiment, the present invention further relates to a method for formulating a therapeutic regimen comprising the steps of: predicting metastasis; and formulating the therapeutic regimen accordingly.

The present invention encompasses the identification of genes which regulate metastasis. Metastasis is a fatal step in the mortality of an organism. The present invention can be used in methods of detection, prevention and treatment of metastasis.

The invention will be further described by the following non-limiting examples. The teachings of all references, patents and web sites referred to herein are incorporated herein by reference in their entirety.

TABLE 1
Enhanced Gene Expression in Metastatic Melanomas

| | Human | | | | | | Mouse | | | | | |
|---|---|---|---|---|---|---|---|---|---|---|---|---|
| | Accession | | | | | | Accession | | | | | Nuc. |
| Gene Name | Number | Ch# | P | M1 | M2 | SM | Number | F0 | F1 | F2 | F3 | Ident. |
| Fibronectin | X02761 | 2 | 1 | 10.1 | 3.2 | 4 | M18194 | A | 2.8 | 2.8 | 2.8 | 93% |
| RhoC | L25081 | 1 | A | 4.7 | 3.1 | 2.8 | X80638 | A | 2.9 | 4.9 | 2.5 | 91% |
| Thymosin β4 | M17733 | X | 1 | 3.3 | 3.6 | 3.5 | W41883 | 1 | 4.1 | 3.5 | 3.5 | 92% |

Gene expression in human A375 melanomas

| t-PA | K03021 | 8 | A | 5.2 | 9.6 | 5.2 | J03520 | A | A | A | A | 81% |
|---|---|---|---|---|---|---|---|---|---|---|---|---|
| Angiopoietin1 | D13628 | 8 | 1 | 4.3 | 9.4 | 3.3 | U83509 | * | | | | |
| IEX-1/Glu96 | S81914 | 6 | 1 | 9.1 | 3.3 | 4.5 | X67644 | 1 | 0.4 | 0.6 | 0.5 | 83% |
| RTP/NDR1 | D87953 | 8 | 1 | 8.6 | 5.4 | 4.7 | U60593 | 1 | A | 0.7 | 1.5 | 86% |
| Fibromodulin | U05291 | 1 | A | 8.3 | 4.7 | 8.2 | X94998 | 1 | 2 | 2 | 1.1 | 90% |
| Hsp70 | M11717 | 1 | 1 | 7.8 | 4.2 | 5 | M20567 | 1 | 2.1 | 1.8 | 1.8 | 80% |
| IL13 Rec., α2 | U70981 | X | 1 | 7.6 | 2.9 | 3.1 | U65747 | * | | | | |
| Sec61β | L25085 | 9 | 1 | 3.8 | 5.3 | 3.2 | ** | | | | | |
| snRNP, poly.pep. C | HG1322- | | 1 | 3.9 | 4.7 | 3.3 | ** | | | | | |
| Collagen 1α2 | Z74616 | 7 | A | 2.5 | 3.6 | 3.6 | X58251 | A | 3.1 | 2.3 | 3.7 | 86% |

| | | | | | | | | |
|---|---|---|---|---|---|---|---|---|
| UBE2I | U45328 | 16 | | 1 | 3.6 | 3.4 | 3.4 | ** |
| KIAA0156 | D63879 | 12 | A | 2.9 | 3.1 | 3.5 | ** | |
| TGFβ superfamily | AB000584 | 19 | 1 | 3.4 | 3.4 | 3 | ** | |

Gene expression in mouse B16 melanomas

| Surfactant Protein C | J03890 | * | | | | | M38314 | A | 32 | 12 | 16 | |
|---|---|---|---|---|---|---|---|---|---|---|---|---|
| Lysozyme M | ** | | | | | | M21050 | A | 20 | 10 | 22 | |
| Matrix Gla Prot | X53331 | 12 | 1 | 3.2 | 4.4 | 1.1 | D00613 | 1 | 12 | 11 | 5.4 | 81% |
| Tsa-1 | ** | | | | | | U47737 | A | 9.7 | 6.1 | 7.2 | |
| Collagen IIIα1 | X06700 | 2 | A | A | A | A | X52046 | A | 8.2 | 5.6 | 5.5 | 89% |
| Biglycan | J04599 | X | A | A | 3.7 | A | L20276 | A | 3.8 | 4.4 | 6.9 | 87% |
| α-catenin | U03100 | 5 | 1 | 1.3 | 1 | 1.9 | X59990 | 1 | 3.4 | 3 | 5.7 | 91% |
| Valosin-cont. prot. | AC004472 | * | | | | | Z14044 | 1 | 3 | 3.9 | 5.9 | |
| ERK-1 | X60188 | 16 | A | A | A | A | Z14249 | 1 | 2.6 | 2.6 | 3 | 85% |

Mouse ESTs

| | | | | | |
|---|---|---|---|---|---|
| α-actinin 1 | AA068062 | 1 | 3.6 | 3.3 | 7.3 |
| calmodulin | AA103356 | A | 4.8 | 6.7 | 5.5 |
| E1F4y | AA002277 | A | 4.7 | 3.2 | 2.6 |
| α-centractin | W48490 | 1 | 2.9 | 3.8 | 3.6 |
| IQGAP1 | AA118739 | A | 3.6 | 3.5 | 3.2 |
| cathepsin s | W13263 | A | 2.8 | 2.8 | 3.1 |
| EF2 | W90866 | 1 | 2.6 | 2.5 | 2.9 |

TABLE 2
Gene Expression in Metastatic
Melanomas Grown as Subcutaneous Tumors

| | Human | | | |
|---|---|---|---|---|
| | Accession | P | SM | SM |
| Gene Name | Number | (sc) | (iv) | (sc) |
| Fibronectin | X02761 | 1 | 4.0 | 9.4 |
| RhoC | L25081 | A | 4.7 | 8.0 |
| Thyrnosin β4 | M17733 | 1 | 3.3 | 2.0 |
| t-PA | K03021 | A | 5.2 | 2.6 |
| Angiopoietin 1 | D13628 | 1 | 3.3 | 3.1 |
| IEX-1 | S81914 | 1 | 4.5 | 10.3 |
| RTP | D87953 | 1 | 4.7 | 2.7 |
| Fibromodulin | U05291 | A | 3.2 | 5.6 |
| Hsp70 | M11717 | 1 | 5.0 | 2.1 |
| IL13 Rec., α2 | U70981 | 1 | 3.1 | 3.2 |
| Sec61β | L25085 | 1 | 3.2 | 3.3 |
| snRNP, polypep. C | HG1322- | 1 | 3.3 | 2.6 |
| collagen Iα2 | Z74616 | A | 3.6 | 1.9 |
| UBE2I | U45328 | 1 | 3.4 | 2.9 |
| KIAA0156 | D63879 | A | 35. | 3.4 |
| TGFβsuperfamily | AB000584 | 1 | 3.0 | 0.6 |

TABLE 3
Pulmonary Metastases

| Cell Line | # of Metastases | # of mice |
|---|---|---|
| A375P | 0,0,0,0,0,1,5,10 | 8 |
| A375P-RhoC | 56,70,>100,>100 | 4 |
| A375M | all >100 | 8 |
| A375M-dnRho | 13,24,29,32 | 4 |

TABLE 4
Cell Proliferation

| | | | | Size of |
|---|---|---|---|---|
| Cell Line | Day 2 | Day 4 | Day 7 | Subcu Tumor |
| A375P | 11.5 ± 1.5 | 54 ± 4.9 | N.D. | 0.50 ± .15 |
| A375P-RhoC | 15.3 ± 1.8 | 66 ± 4.1 | N.D. | 0.45 ± .14 |
| A375P-RhoA | 12.5 ± 1.5 | 48 ± 3.6 | N.D. | N.D. |
| A375M | 19 ± 0.6 | 58 ± 2.3 | 128 ± 10 | 0.42 ± .13 |
| A375M-dnRho | 10 ± 4.5 | 58 ± 8.4 | 135 ± 8.6 | 0.41 ± .12 |

TABLE 5
ADDITIONAL HETASTASIS GENES

Additional Genes
Cytochrome c-1
Peptidylprolyl isomerase B (cyclophilin B)
CD58 antigen (lymphocyte function-associated antigen 3)
Splicing factor, SF1-Bo isoform
Putative serine/threonine protein kinaae (Y10032)
CID protein
Annexin ⊥ (lipocortin)
TIMP-3
Tubby homolog
Protein tyrosine phosphatase, non-receptor type 12
NAD-dependent methylene tetrahydrofolate dehydrogenase cyclohydrolase
Tissue inhibitor of metalloproteinase 2
Syntaxin-16C
Clone RES4-24A
Protein tyrosine phosphatase PTPCAAX1
Mucin
GIF
CDC28 protein kinase 2
Squalene epoxidase
OZF
CD9 antigen
Arginine-rich protein (ARP)
Calponin 3, acidic
Metalloproteinase inhibitor 3 precursor
Small nuclear ribonucleoprotein polypeptide B
Lysyl hydroxylase
Induced myeloid leukemia cell differentiation protein MCL1
Chromosome 17q21 mRNA clone LF113
Putative transmembrane protein (nma)
Laminin, gamma 1
Chromosome segregation gene homolog CAS
KIAA0156 gene
Chondroitin sulfate proteoglycan 2 (versican)
RagA protein
Lumican
TAPA-1
KIAA0170
Cytoplasmic Dynein light chain 1
Insulin-like growth factor binding protein 3 precursor
Factor VII serine protease precursor
Laminin, alpha 4
Sp1 transcription factor
Apolipoprotein D
Elastin
Annexin II
RP3
MUC18
BAT3
SPARC/osteonectin
Translation elongation factor 1 gamma
Eukaryotic translation initiation factor 4A
Superoxide dismutase 3
Thymosin beta-10
autotaxin
Succinate dehydrogenase, iron sulfur subunit
Heterogeneous nuclear ribonucleoprotein A2/B1

### EXAMPLES

METHODS:

Cell Lines

The A375 (ATCC #CRL-1619) and B16 (ATCC# CRL-6322) cell lines were grown on plastic in monolayer cultures and maintained in DMEM supplemented with 10% FBS, 2 mM sodium pyruvate, MEM non-essential amino acids, L-glutamine and vitamins. The cells were harvested by trypsinization and washing of the suspended cells in PBS. The suspension was diluted to yield 2.5×106 cells per ml for A375 cells and 2.5×10⁵ cells per ml for B16 cells.

Experimental Metastasis Assays

A375 cells were injected either intravenously (0.2 ml) into the lateral tail vein or subcutaneously (0.1 ml) into the dorsal flank of nude mice, and B 16 cells were injected into syngeneic C57BL/6 mice. Three (for B16) to eight (for A375) weeks after injection the mice were sacrificed; the lungs were removed and washed, and the pulmonary metastases on the lung surface were counted under a dissecting microscope. Metastatic nodules were removed aseptically, minced, and grown in vitro, or snap-frozen in liquid nitrogen to purify RNA.

Tumors and Tumor-Derived Cell Lines

A375M1, M2, and SM lines were selected using the experimental metastasis assay for their enhanced ability to form experimental pulmonary metastases (Fidler, 1973). Line M1 was derived from metastases isolated from mice injected intravenously with the A375P cells, line M2 from mice injected with A375M1 cells, and line SM was a gift from Dr. I. Fidler (MD Anderson Cancer Center) and was derived by an identical selection procedure (Kozlowski et al, 1984). B16 lines were derived in an identical manner, with F1 cells derived from B16F0 cells, F2 from B16F1 cells, and F3 from B16F2 cells. The A375M cell line is a pool of cells from A375M1, M2, and SM cells. A375P and A375M cells used in retroviral gene transfer studies were transfected with a plasmid containing the ecotropic receptor (a gift of Dr. H. Lodish (Whitehead Institute)) and selected for neomycin resistance.

Array Hybridization

Total RNA was prepared with a Qiagen RNeasy mini-kit according to the manufacturer's instructions. cRNA for hybridization was prepared essentially as described (Fambrough et al., 1999). Oligonucleotide arrays (GeneChip®, Affymetrix, Santa Clara, Calif.) composed of 6800 human or 6500 mouse genes and ESTs were used for hybridization according to the manufacturer's instructions. Arrays were scanned using a Molecular Dynamics confocal scanner and analyzed using GeneChip®3.0 software (Affymetrix). Intensity values were scaled so that the overall fluorescence intensity of each chip of the same type was equivalent.

Criteria for Selecting Induced Genes

For a gene to be selected as induced as described herein, it has to be expressed in all three metastatic samples (either M1, M2, and SM or F1, F2, and F3) at least 2.5-fold higher than in the poorly metastatic sample (either P or F0), done in duplicate. Where expression in the poorly metastatic sample was below baseline (arbitrarily set at 20, the point below which changes in expression could be determined with high confidence), it was determined to be absent and was set to 20. Reproducibility experiments were used to define the 2.5-fold expression threshold; at this threshold a 0.04% false positive rate (one false positive in 2500 genes) was achieved for a duplicate sample.

Cloning

The human fibronectin (Genbank accession number X02761), rhoC (L25081), and thymosin β4 (M17733) genes were cloned using a Zero Blunt TOPO PCR Cloning Kit (In Vitrogen) according to the manufacturers instructions. PCR fragments for cloning were generated with vent polymerase as follows: for fibronectin, a 425 base pair (bp) fragment (nucleotides 6848 to 7273) was synthesized using the primers GTCCCGAAGGCACTACT (SEQ ID NO: 1) and ATCCCAAACCAAATCTTA (SEQ ID NO: 2), for rhoC a 626 bp fragment (nucleotides -3 to 623) was synthesized using the primers ACCATGGCTGCAATCCGAAAGAAG (SEQ ID NO: 3) and AAGGGAGGGGGCATGTAGGAAAAG (SEQ ID NO: 4); and for thymosin β4 a 405 bp fragment (nucleotides −28 to 377) was synthesized using the primers CGCCTCGCTTCGCTTTTC (SEQ ID NO: 5) and CACCCCACTTCTTCCTTCACCA (SEQ ID NO: 6). For rhoC and thymosin β4, the PCR fragments contain the entire coding region and significant 3′ sequence. After cloning into the pCR-BluntII TOPO vector, the PCR products were sequenced to confirm the sequence obtained.

RNAse Protection Assays

RNAse protection was performed as previously described (Whittaker and DeSimone, 1993). The fibronectin probe was created by digesting the pCR-BluntfII-fibronectin vector with Mfel. This creates a 343-nucleotide protected fragment. The rhoC probe was created by digesting the pCR-BluntII-rhoC vector with Xmn1, creating a 310-nucleotide protected fragment. The thymosin β4 probe was created by digesting the pCR-BluntII-thymosin β4 vector with Dral, creating a 133-nucleotide protected fragment. The β-actin control template was purchased from Ambion. Autoradiographic films were quantitatively analyzed using an Is-1000 Digital Imaging System (Alpha innotech Corporation).

Subcloning and Retroviral Gene Transfer

An EcoR1 fragment of pCR-BluntII-rhoC containing the entire coding region of human rhoC was inserted into the EcoR1 site of the retroviral bicistronic expression vector pMX-IRES-GFP (pMIG (Liu et al., 1997)) containing enhanced green fluorescent protein (GFP) as an expression marker. An EcoR1 fragment of pEXV-rhoA or pEXV-N19rhoA (a dominant-negative rho mutant (dnRho)) was inserted into the EcoR1 site of pMIG. pMIG-rhoC, pMIG-rhoA, and pMIG-dnRho were transfected into 293T cell-derived retroviral producer lines (Phoenix cells) as described (see website at www.stanford.edu/group/nolan/). Seventy-two hours after transfection, virus supernatant was collected. Then 5×10⁵ A375P or M cells were infected with 0.5 ml of virus supernatant in the presence of polybrene for 6 hours at 33° C. and fresh media was added. Forty-eight hours post-infection the cells were sorted by FACStar (Becton-Dickinson) according to their GFP levels and were called A375P-rhoC, A375P-rhoA, or A375M-dnRho cells. A375P-rhoC and A375P-rhoA cells expressed similar levels of GFP.

Proliferation Assay

One ml of A375 cells suspended in media containing 1% or 10% FBS were plated at 5×10⁴ cells per well on six well Falcon plates (35 mm per well). Cells were trypsinized and counted on days 2, 4 and 7.

Chemotaxis and Invasion Assays

Cell migration and invasion assays were performed using 6.5 mm 8.0 Jim pore size Transwells inserts (Costar Corporation) or 6.4 mm Biocoat Matrigel Invasion Chambers (Becton-Dickinson), respectively. A375 cells were suspended in serum-free media at 2×105 cells per ml; 0.25 ml of cell suspension was added to the upper chamber and 0.75 ml of media containing 10% FBS was added to the lower chamber. After 16 hours (for chemotaxis) or 48 hours (for invasion) of incubation at 37° C., all non-migrant cells were removed from the upper face of the membrane with a cotton swab. Migrant cells attached to the lower face were rinsed in PBS, fixed for 10 minutes in 4% paraformaldehyde/PBS, and stained with 0.1% crystal violet. Stained cells were then photographed and the crystal violet stain extracted with 10% acetic acid. Absorbance at 600 nm was then determined. Each data point represents the average of four (for chemotaxis) or three (for invasion) individual experiments, done in duplicate, and error bars represented the standard error of the mean.

Immunofluorescence

Adherent cells were fixed, permeabilized, and stained as described previously (Clark et al, 1998).

RESULTS

In vivo Selection of Metastatic Tumor Cells

When nude mice were injected intravenously with the amelanotic human A375P tumor cells, relatively few pulmonary metastases were observed (see FIG. 2A and Table 3). Table 3 shows the number of pulmonary metastases identified on the surface of the lungs of mice injected with A375P, A37P-RhoC, A375M, or A375M-dnRho cells. However, when these metastases were dissected free of the lungs and the cells grown in tissue culture, the resulting cells showed enhanced metastatic capacity, confirming that highly metastatic cells can be selected from a heterogenous population of poorly metastatic tumor cells (Kozlowski et al., 1984). Furthermore, if successive metastases (designated M1 and M2) were isolated, expanded in tissue culture, and re-introduced into host mice as shown in FIG. 1, significantly more pulmonary metastases were observed (FIG. 2B and Table 3). When the mouse B16F0 melanoma cells were subjected to this same in vivo selection scheme, highly metastatic pulmonary tumors (designated F1, F2, and F3) were isolated as previously described for this cell line (Fidler, 1973). When the poorly metastatic A375P or B16F0 and the in vivo-selected metastatic A375 or B16 cells were grown as subcutaneous tumors, there was no observable difference in tumor size (Table 4), suggesting selection for a difference in the metastatic, but not tumorigenic, properties of the melanomas. Table 4 illustrates the results of cell proliferation studies. A375 cells were plated at 5×10⁴ cells on Day 0. Cells were trypsinized and counted on days 2, 4, and 7, and the results are expressed as cell numbers (×10⁴)±SEM (N=3). Subcutaneous tumors, examined 42 days after injection of tumor cells, were measured in three dimensions and the results expressed in cm³.

Metastasis Genes Identified by Genomic Analysis

RNA extracted from these pulmonary metastases (or the parental A375P and B16F0 lines grown as subcutaneous tumors) was then used in preparation of the cRNA targets which were hybridized to the oligonucleotide microarrays to determine the array of differentially expressed genes (FIG. 1).

In Table 1, genes whose expression is enhanced in pulmonary metastases (M1, M2, SM, F1, F2, or F3) are compared to poorly metastatic cells (P and F0) grown as subcutaneous tumors. The values for P and F0 are the average of two experiments performed with subcutaneous tumors from two mice injected with A375P or B16F0 cells. Data is presented as fold expression compared to the poorly metastatic tumors. When expression was below baseline, the expression was marked as absent (A) and was arbitrarily set at 20. “*: ” means a mouse or human gene homologue exists in the UNIGENE database but was not part of the oligonucleotide probe set. “**: ” means no gene homologue was found in the UNIGENE database. Mouse expressed sequence tags (ESTs) are noted in italics and are named according to the gene to which they show the greatest sequence similarity. “Ch#: ” is the human chromosome where the gene resides. “Nuc Indent: ” means the percentage of nucleotides identical between the human and mouse homologues, as determined by BLAST search. The listed accession number is the GenBank entry from which the oligonucleotide probe sequences were drawn.

The data shown in the top half of Table 1 is the subset of genes expressed at consistently higher levels in the pulmonary metastases (M1, M2, and SM) when compared to the poorly-metastatic A375P tumor. Genes expressed at higher levels in the pulmonary metastases generated from the mouse B16 line (F1, F2, and F3) when compared to the poorly-metastatic B16F0 tumor are shown in the lower half of Table 1. Three genes, fibronectin, rhoC, and thymosin β4, were expressed at higher levels in all three metastases selected from both the human A375 and mouse B16 cell lines, suggesting that their altered expression may be important for tumor metastasis. Enhanced expression of these three genes in the pulmonary metastases was confirmed by RNAse protection (FIG. 2).

To ensure that the enhanced expression of these genes in the pulmonary metastases was not due solely to the microenvironment in which the metastatic cells were growing, the metastatic A375SM cells were injected subcutaneously, and the expression profile of this tumor was compared to the subcutaneous A375P tumor. Table 2 shows genes whose expression is enhanced in metastatic tumor cells (SM) grown as pulmonary metastases (iv) and subcutaneous tumor (sc). The data is presented as in Table 1. As shown in Table 2, 15 of the 16 genes continued to show enhanced expression when the metastatic A375 cells were grown as a subcutaneous tumor, suggesting that the expression of these genes is intrinsic to the metastatic cells. It should be noted, however, that the tumor microenvironment may play a role in regulating the absolute level of gene expression. Table 5 also shows the genes (gene products) which passed two of the three stringency criteria set as described herein; thus, the genes listed in Table 5 are also considered metastasis genes.

Fibronectin is an extracellular glycoprotein that serves as a ligand for the integrin family of cell adhesion receptors. RhoC is a member of the Rho GTPase family that has been shown to regulate numerous cellular functions, most notably cytoskeletal organization in response to extracellular factors (van Aelst and D'souze-Schorey, 1997). Thymosin β4 is an actin-sequestering protein that regulates actin polymerization that has not been directly implicated in metastasis. Other regulators of the cytoskeleton also appear on the list, including ESTs for α-actin 1 and α-centractin, and α-catenin, an intracellular component of cadherin-mediated cell-cell adhesions. Cadherins are linked to the actin-based cytoskeleton through α-catenin (Ranscht, 1994). The altered expression of so many genes whose products regulate the actin cytoskeleton either directly or indirectly suggests an important role for cytoskeletal organizaiton in tumor metastasis.

Prominent on the list in Table 1 are several genes that encode extracellular matrix proteins, as well as molecules that regulate their assembly. In addition to fibronectin, two collagen subunits (the α2 subunit of type 1 collagen and the α1 subunit of type III colagen), the matrix Gla protein, fibromodulin, and biglycan also are expressed at higher levels in the metastatic melanomas. Several other genes implicated in events essential for metastasis include angiopoietin 1, a regulator of angiogenesis, and tissue plasminogen activator (tPA), which may serve as a catalyst to activate proteolytic cascades involved in tumor cell invasion. In addition, several genes on the list have yet to be identified as playing a role in tumor metastasis, although their altered expression in this system suggests that they too may control events essential to metastasis.

Several genes that do not appear on this list are conspicuous in their absence. Several metastasis suppressor genes, such as nm23, KiSS 1, and CD82, have been identified in other studies and shown to be capable of inhibiting tumor metastasis (Fidler and Radinsky, 1996). In this study all three of these genes were absent in both the parental A375 tumors and in the metastases, suggesting that while expression of these genes may inhibit metastasis, lack of their expression is not sufficient for metastasis. Other genes not found in Table 1 but whose expression correlates with melanoma metastasis in previous studies include the Met tyrosine kinase receptor, matrix metalloproteinases (MMPs) such as MMP2, and the β3-integrin subunit (Jeffers et al., 1996; Chambers and Mtrisian, 1997; Albelda et al., 1990). In the B16 tumors, Met expression was higher in two of the three metastases but its expression was not detected in any of the A375 tumors, suggesting that its expression is not essential for these tumors to metastasize. Expression of MMP2 and of the β3-integrin subunit was not significantly higher in any of the three metastases, but their expression in both the parental and metastatic tumors may be sufficient to allow the tumor cells to metastasize.

RhoC is Essential For Metastasis

Having uncovered 32 genes and ESTs whose expression pattern suggests a role in metastasis, the role of these genes in this process was investigated. Because of its elevated expresion in metastases derived from both tumor cell lines, rhoC was chosen to confirm the hypothesis that these expression studies will identify genes essential for metastasis. The full-length human rhoC gene was cloned, subcloned into a retroviral vector, and introduced into a retroviral packaging cell line. Retroviral particles were used to infect the poorly metastatic A375P cells, and cells expressing high levels of rhoC were selected by FACS. These cells, designated A375P-rhoC were subjected to the experimental metastasis assay. As seen in FIG. 3C and Table 3, rhoC dramatically enhanced metastasis in this system.

Next it was determined if one could inhibit metastasis by expressing an inhibitory form of rho in the highly metastatic A375M cells. This work took advantage of a know dominant-inhibitory rho mutant (N19rho) (Quilliam et al., 1995); this mutant is analogous to the N17ras mutant that has been shown to block ras signalling (Feig and Cooper, 1988). Ras dominant-negatives are actually antagonists of the guanine-nucleotide exchange factors (GNEFs) for ras, rather than ras itself (Quilliam et al., 1995). These results suggest that a dominant-negative rhoA would antagonize rho GNEFs, thereby inhibiting rhoC. Expression of N19rhoA in the A375M cells dramatically inhibited the generation of metastases when these cells were subjected to the experimental metastasis assay (Table 3), suggesting that rho activity is necessary, and rhoC is sufficient, for metastasis.

RhoC Enhances Invasive Phenotype

Having established that rhoC is both necessary and sufficient for metastasis, further work was done to identify how rhoC regulates the ability of tumor cells to metastasize. As described above, tumor cells must complete a complex series of steps to metastasize. One of the most basic steps is cell growth. Rho GTPases are known to control several aspects in growth control (Van Aelst and D'Souza-Schorey, 1997), so it was possible that rhoC might control tumor metastasis by regulating cell proliferation. To test this hypothesis the A375P, A375P-rhoC, A375M and A375M-dnRho cells were subjected to both an in vitro proliferation and in vivo tumorigenesis assay. As shown in Table 4, proliferation in either assay was not significantly changed by altering RhoC expression or rho activity, suggesting that rhoC regulates metastasis by a mechanism other than by controlling cell proliferation.

Another function of Rho-family GTPases is to control cytoskeletal organization in response to extracellular factors (Van Aelst and D'Souza-Schorey, 1997). Cytoskeletal proteins are known effectors for events essential for cell motility (Lauffenburger and Horwitz, 1996), another process implicated in metastasis. Therefore, rhoC may control metastasis by regulating cell motility. Metastatic A375M cells were more migratory (FIG. 2A) and more invasive (FIG. 2B) than the poorly metastatic A375P cells. Furthermore, rhoC could enhance the migratory and invasive capacity of the A375P cells, while dnRho inhibited motility and invasion of the A375M cells, suggesting that rhoC may regulate metastasis by controlling cytoskeletal events essential for motility. In support of this, it was observed that rhoC could induce in A375P cells an elongated morphology similar to that observed in A375M cells, while dnRho expression inhibited this morphology. Metastatic capacity did not correlate with another morphological difference noted in the A375M cells, the serum-induced formation of filopodia, suggesting that these structures may be dispensible for metastasis.

While this invention has been particularly shown and described with references to preferred embodiments thereof, it will be understood by those skilled in the art that various changes in form and details may be made therein without departing from the scope of the invention encompassed by the appended claims.

References

Albelda, S. M. et al. Integrin distribution in malignant melanoma: association of the β3 subunit with tumor progression. Cancer Res. 50 6757-6764 (1990).

Bao. L. et al. Thymosin β15: a novel regulator of tumor cell motility upregulated in metastatic prostate cancer. Nature Medicine 2 1 2-i3-1328 (1996).

Chambers, A. F. & Martisian, L. M. Changing views of the role of matrix metalloproteinases in metastasis. J. Natl. Cancer Inst. 89 1260-1270 (1997).

Chen. L., O'Bryan, J. P., Smith, H. S.. & Liu. E. Overexpression of matrix Gla protein mRNA in malignant human breast cells: isolation by differential cDNA hybridization. Oncogene 5 1391-1395 (1990).

Clark, E. A., King, W. G., Brugge, J. S., Symons, M., & Haynes R. O. Integrin-mediated signals regulated by members of the rho family of GTPases. J. Cell Biol. 142 573-586 (1998).

Fambrough, D., McClure, K., Kazlauskas, A., & Lander, E. S. Diverse signaling pathways activated by growth factor receptors induce broadly overlapping, rather than independent, sets of genes. Cell 97 727-741 (1999).

Feig. L. A. & Cooper. G. M. Inhibitor of NIH-3T3 cell proliferation by a mutant ras protein with preferentail affinity for GDP. Mol Cell Biol. 8 3235-3243 (1988).

Fidler, I. J. Selection of successivc tumour lines for metastasis. Nature 242 148-149 (1973).

Fidler, I. J. & Radinsky, R. Search for genes that suppress cancer metastasis. J. Natl. Cancer Inst. 8 1700-1703 (1996).

Fodon. S. P. A. Massively parallel genomics. Science 277 393-395 (1997).

Habets, G. G. M. et al. Identification of an invasion-inducing gene, Tiam-1, that encodes a protein with homology to GDP-GTP exchangers for rho-like proteins. Cell 77 537-549 (1994).

Hall, A. K. Differential expression of thymosin genes in human tumors and in the developing human kidney. Int. J. Cancer 48 672 (1991).

Humphries, M. J., Olden, K. & Yamada, K. M. A synthetic peptide from fibronectin inhibits experimental metastasis of murine melanoma cells. Science 233 467-469 (1986)

Itoh, K. et al. An essential part for Rho-associated kinase in the transcellular invasion of tumor cells. Nature Med. 5 221-225 (1999).

Jeffers, M., Rong, S. & Vande Woude, G. F. Enhanced tumorigenicity and invasion-metastasis by hepatocyte growth factor/scatter factor-Met signalling human cells concomtant with induction of the urokinase proteolysis network. Mol. Cell. Biol. 16 1115-1125 (1996).

Kozlowski, J. M., Hart, I. R., Fidler, I. J. & Hanna, N. A human melanoma cell line heterogenous with respect to metastic capacity in athymic nude mice. J. Natl. Cancer Inst. 72 913-917(1984).

Laufenburger, D. A. & Horwitz, A. F. Cell migration; a physically integrated molecular process. Cell 84 359-369 (1996).

Liu, N., et al. Transforming growth factor β-induced phosphorylation of Smad3 is required for growth inhibition and transcription induction in epithelial cells. Proc. Natl. Acad. Sci. USA 94 10669-10674 (1997).

Mamotis, A. J. et al. Vascular channel formation by human melanoma cells in vivo and in vitro: vasculogenic mimicry. Am. J Pathol. 155 739-752 (1999).

Qiu, R. G., Chen, J., McCormick, F., & Symons, M. A role for rho in ras transformation. Proc. Natl. Acad. Sci. USA 92 11781-11785 (1995)

Quillra, L. A., Khosravi-Far. R. Huff, S. Y., & Der. C. J. Guanine nucleotide exchange factors: acuators of the Ras superfamily of protiens. Bioessays 17 395-404 (1995).

Rans, M. B. Cadherins and catenins; interactions and functions in embryonic development. Curr. Opin. Call Biol. 6 740-746 (1994)

Roberts, D. D. Regulation of tumor growth and metastasis by thrornbospondin-1. FASEB J 10 1183-1191 (1996).

Schonherr, E. et al. Interation of biglycan with type 1 collagen. J. Biol. Cliem. 270 2776-2783 (1995).

Sherman, L., Sleeman, J., Herrlich, P., & Ponta, H. Hyaluronate receptors: key players in growth, differentiation, migration and tumor progression. Curr. Opin. Cell Biol. 6:726-733 (1994).

Suwa, H. et al. Overexpression of the rhoCgene correlates with progression of ductal adococarcinoma of the pancreas. Br. J. Cancer 77 147-152(1998).

Svensson, L. et al. Fibromodulin-null mice have abnormal collagen fibrils, tissue organization, and altered lumican depostion in tendon. J. Biol. Chem. 274 9636-9647 (1999).

Van elst, L., & D'Souza-Schorey, C. Tho GTPases and signaling networks. Genes Dev. 11 2295-2322 (1997).

Van oorden, C. J. F., Meade-Tollin, L. C. & Bosman, F. T. Metastasis. American Scientist 86 130-141 (1998).

Vermeulen, S. J. et al. Transition from the noninvasive to the invasive phenotype and loss of a-catenin in human colon cancer cells. Cancer Res. 55 4722-4728 (1995).

Vousden, K. H., Eccles. S. A., Purvies, J. & Marshall, C. J. Enhanced spontaneous metastasis of carcinoma cells transfected with an activated c-Ha-ras-1 gene. Int. J. Cancer 37 425-433 (1986).

Welch, D. R. et al. Microcell-mediated transfer of chromosome 6 into metastatic human C8161 melanoma cells suppresses metastasis but does not inhibit tumorigenicity. Oncogene 9 255-262 (1994).

Weterman, M. A. J., van Muigen, G. N. P., Ruter, D. J., & Bloemers, H. P. J. Thymosin β-10 expression in melanoma cell lines and melanocytic lesions; a new progression marker for human cutaneous melanoma. Int. J. Cancer 53 278-284 (1993).

Whittake, C. A., & DeSimome, D. W. Integrin αsubunit mRNAs are differentially expressed in early Xenopus embryos. Development 117 1239-1249 (1993).

Zhans, L. et al. Gene expression profiles in normal and cancer cells. Science 276 1268-1272 (1997).

What is claimed is:

1. A method of inhibiting metastasis in a mammal, comprising administering to a mammal in need thereof an effective amount of an agent which alters the actin- based cytoskeleton of one or more tumor cells in the mammal thereby inhibiting metastasis.

2. A method according to claim 1, wherein the mammal is a human.

3. A method according to claim 1, wherein the agent inhibits the actin-based cytoskeleton.

4. A method of inhibiting metastasis in a mammal, comprising administering to a mammal in need thereof an effective amount of an agent wherein said agent inhibits the activity of one or more genes selected from the group consisting of the genes encoding fibronectin, RhoC, thymosin β4, t-PA, angiopoietin 1, IEX-1/Glu96, RTP/NDR1, fibromodulin, Hsp70, IL13 Rec. α2, Sec61β, snRNP polypeptide C, collagen Iα2, UBE21, KIAA0156, TGFβ superfamily, surfactant protein C, lysozyme M, matrix Gla protein, Tsa- 1, collagen IIIα1, biglycan, α-catenin, valosin-containing protein, ERK-1, α-actinin 1, calmodulin, EIF4γ, α-centractin, IQGAP 1, cathepsin S and EF2 such that metastasis is inhibited.

5. A method according to claim 4, wherein the agent inhibits the activity of the gene directly.

6. A method according to claim 4, wherein the agent inhibits the activity of the gene by inhibiting the activity of a downstream effector of the gene.

7. A method according to claim 4, wherein the gene encodes RhoC.

8. A method according to claim 1, wherein the mammal has one or more non- metastatic tumors and wherein the agent alters the actin-based cytoskeleton of one or more cells of the tumor.

9. A method according to claim 8, wherein the mammal has one or more non- metastatic conditions selected from the group consisting of melanoma, breast cancer, ovarian cancer, prostate cancer, lung cancer, bone cancer, throat cancer, brain cancer, testicular cancer, liver cancer, stomach cancer, pancreatic cancer, and combinations thereof.

10. A method according to claim 1, wherein the agent is selected from the group consisting of nucleic acid molecules, antibodies, peptides, organic molecules, inorganic molecules, and combinations thereof.

11. A method according to claim 10, wherein the nucleic acid molecules are selected from the group consisting of one or more antisense molecules and nucleic acid molecules encoding one or more dominant negative forms of a gene product.

12. A method of predicting the likelihood of development of a metastatic condition in a mammal, comprising the steps of:
a) obtaining a biological sample from a mammal to be tested;
b) determining the level of one or more gene products which alter the actin- based cytoskeleton of one or more tumor cells in the mammal; and
c) comparing the level determined in (b) with an appropriate control, wherein if the level determined in (b) is greater than the level of the gene product in the control, then the mammal has an increased likelihood of developing a metastatic condition.

13. A method according to claim 12, wherein the mammal is a human.

14. A method of predicting the likelihood of development of a metastatic condition in a mammal, comprising the steps of:
a) obtaining a biological sample from a mammal to be tested;
b) determining the level of one or more gene products selected from the group consisting of fibronectin, RhoC, thymosin , β4, t-PA, angiopoietin 1, IEX-1/Glu96, RTP/NDR1, fibromodulin, Hsp70, IL13 Rec. α2, Sec61β, snRNP polypeptide C, collagen Iα2, UBE21, KIAA0156, TGFβ superfamily, surfactant protein C, lysozyme M, matrix Gla protein, Tsa-1, collagen IIIα1, biglycan, α-catenin, valosin-containing protein, ERK- 1, α-actinin 1, calmodulin, EIF4γ, α-centractin, IQGAP1, cathepsin S, or EF2, in one or more tumor cells in the mammal; and
c) comparing the level determined in (b) with an appropriate control, wherein if the level determined in (b) is greater than the level of the gene product in the control, then the mammal has an increased likelihood of developing a metastatic condition.

15. A method according to claim 14, wherein the gene product is RhoC.

16. A method according to claim 12, wherein the control is a sample from a normal mammal.

17. A method according to claim 12, wherein the metastatic condition is selected from the group consisting of metastatic forms of melanoma, breast cancer, ovarian cancer, prostate cancer, lung cancer, bone cancer, throat cancer, brain cancer, testicular cancer, liver cancer, stomach cancer, pancreatic cancer, and combinations thereof.

18. A method according to claim 4, wherein the agent inhibits a gene product at the level of transcription, translation, or protein activity.

19. A method according to claim 12, wherein the biological sample is a blood sample or a cell sample from a tumor in the mammal.

20. A method of identifying an agent which regulates metastasis of a tumor cell, comprising the steps of:
a) contacting one or more tumor cells with an agent to be tested; and
b) determining the level of one or more gene products which regulate the actin-based cytoskeleton in the cell, wherein if the level of the gene product is altered in the presence of the agent as compared with the level of the gene product in the absence of the agent, then the agent regulates metastasis of a tumor cell.

21. A method of identifying an agent which regulates metastasis of a tumor cell, comprising the steps of:
a) contacting one or more tumor cells with an agent to be tested; and
b) determining the level of one or more gene products selected from the group consisting of fibronectin, RhoC, thymosin β4, t-PA, angiopoietin 1, IEX-1/Glu96, RTP/NDR1, fibromodulin, Hsp70, IL13 Rec. α2, Sec61β, snRNP polypeptide C, collagen Iα2, UBE21, KIAA0156, TGFβ superfamily, surfactant protein C, lysozyme M, matrix Gla protein, Tsa-1, collagen IIIα1, biglycan, α-catenin, valosin-containing protein, ERK-1, α-actinin 1, calmodulin, EIF4γ, α-centractin, IQGAP 1, cathepsin S and EF2. which regulate the actin-based cytoskeleton in the cell, wherein if the level of the gene product is altered in the presence of the agent as compared with the level of the gene product in the absence of the agent, then the agent regulates metastasis of a tumor cell.

22. A method according to claim 21, wherein the gene product is RhoC.

23. A method of inhibiting metastasis in a mammal, comprising administering to a mammal in need thereof an effective amount of an agent which alters the actin-based cytoskeleton of one or more tumor cells in the mammal, wherein the agent is identified by a method according to claim 20.

24. A method of inhibiting metastasis in a mammal, comprising administering to a mammal in need thereof an effective amount of an agent which alters the expression of a gene which regulates metastasis in one or more tumor cells in the mammal, wherein the agent is identified by a method according to claim 21.

25. A method according to claim 24, wherein the gene product involved in metastasis is selected from the group consisting of fibronectin, RhoC and thymosin β4.

26. A method of inhibiting metastasis in a mammal, comprising administering to a mammal in need thereof an effective amount of an agent which alters the expression of rhoC in one or more tumor cells in the mammal thereby inhibiting metastasis.

27. A method of predicting the likelihood of development of a metastatic condition in a mammal, comprising the steps of:
a) obtaining a biological sample from a mammal to be tested;
b) determining the level of one or more gene product which regulates metastasis in one or more tumor cells in the mammal; and
c) comparing the level determined in (b) with an appropriate control, wherein if the level determined in (b) is greater than the level of the gene product in said control sample, then the mammal has an increased likelihood of developing a metastatic condition.

28. A method according to claim 27, wherein the gene products involved in metastasis is selected from the group consisting of fibronectin, RhoC, thymosin β4, t-PA, angiopoietin 1, IEX-1/Glu96, RTP/NDR1, fibromodulin, Hsp70, IL13 Rec. α2, Sec61β, snRNP polypeptide C, collagen Iα2, UBE21, KIAA0156, TGFβ superfamily, surfactant protein C, lysozyme M, matrix Gla protein, Tsa-1, collagen IIIα1, biglycan, α-catenin, valosin-cont. prot., ERK-1, α-actinin 1, calmodulin, EIF4γ, α-centractin, IQGAP1, cathepsin S and EF2.

29. A method of predicting the likelihood of development of a metastatic condition in a mammal, comprising the steps of:
a) obtaining a biological sample from a mammal to be tested;
b) determining the level of rhoC gene product in one or more tumor cells in the mammal; and
c) comparing the level determined in (b) with the level of rhoC gene product in an appropriate control, wherein if the level determined in (b) is greater than the level of the rhoC gene product in said control, then the mammal has an increased likelihood of developing a metastatic condition.

30. A method of identifying an agent which regulates metastasis of a tumor cell, comprising the steps of:
a) contacting one or more tumor cells with an agent to be tested; and
b) determining the level of one or more gene products which regulates metastasis in a tumor cell, wherein if the level of the gene product is altered in the presence of the agent as compared with the level of the gene product in the absence of the agent, then the agent regulates metastasis of a tumor cell.

31. A method according to claim 30, wherein the gene which regulates metastasis is selected from the group consisting of fibronectin, RhoC, thymosin β4, t-PA, angiopoietin 1, IEX-1/Glu96, RTP/NDR1, fibromodulin, Hsp70, IL13 Rec. α2, Sec61β, snRNP polypeptide C, collagen Iα2, UBE21, KIAA0156, TGFβ superfamily, surfactant protein C, lysozyme M, matrix Gla protein, Tsa-1, collagen IIIα1, biglycan, α-catenin, valosin-cont. prot., ERK-1, α-actinin 1, calmodulin, EIF4γ, α-centractin, IQGAP1, cathepsin S and EF2.

32. A method of identifying an agent which regulates metastasis of a tumor cell, comprising the steps of:
a) contacting one or more tumor cells with an agent to be tested; and
b) determining the level of rhoC gene product, wherein if the level of rhoC gene product is altered in the presence of the agent as compared with the level of rhoC gene product in the absence of the agent, then the agent regulates metastasis of a tumor cell.

33. A method of identifying an agent which inhibits metastasis of a tumor cell, comprising the steps of:
a) contacting one or more tumor cells with an agent to be tested; and
b) determining the level of rhoC gene product, wherein if the level of rhoC gene product is decreased in the presence of the agent as compared with the level of rhoC gene product in the absence of the agent, then the agent inhibits metastasis of a tumor cell.

34. A method of identifying an agent which increase metastasis of a tumor cell, comprising the steps of:
a) contacting one or more tumor cells with an agent to be tested; and
b) determining the level of rhoC gene product, wherein if the level of rhoC gene product is increased in the presence of the agent as compared with the level of rhoC gene product in the absence of the agent, then the agent increases metastasis of a tumor cell.

35. A method for formulating a therapeutic regimen comprising the steps of:
a) predicting metastasis according to claim 27; and formulating the therapeutic regimen accordingly.
